(19) United States
(12) Patent Application Publication
(10) Pub. No.: US 2024/0092870 A1
(43) Pub. Date: Mar. 21, 2024

(54) METHODS OF DETERMINING PROTEIN REDUCTION SUSCEPTABILITY

(71) Applicant: Sanofi, Paris (FR)

(72) Inventors: Antonio Barberio, Cambridge, MA (US); Jiuyi Lu, Cambridge, MA (US)

(21) Appl. No.: 18/241,669

(22) Filed: Sep. 1, 2023

Related U.S. Application Data
(60) Provisional application No. 63/497,629, filed on Apr. 21, 2023.
(60) Provisional application No. 63/406,994, filed on Sep. 15, 2022.

(30) Foreign Application Priority Data
Mar. 10, 2023 (EP) 23315044.0

(51) Int. Cl. C07K 16/00

(52) U.S. Cl. CPC C07K 16/00; C07K 2317/14; C07K 2317/90

(57) ABSTRACT

Provided herein are methods of determining the reduction susceptibility of a protein and methods of adjusting the culturing condition of the protein based on the reduction susceptibility.

### CROSS-REFERENCE TO RELATED APPLICATIONS

This application claims priority to U.S. Provisional Patent Application Ser. No. 63/406,994, filed Sep. 15, 2022, and U.S. Provisional Patent Application Ser. No. 63/497,629, filed Apr. 21, 2023; the entire contents of which are herein incorporated by reference.

### TECHNICAL FIELD

The present disclosure relates to methods of determining the reduction susceptibility of a protein and methods of adjusting the culturing condition of the protein based on the reduction susceptibility.

### BACKGROUND

Antibody reduction is a growing area of concern and study in the cell culture process development field. Antibody reduction refers to the process by which the disulfide bonds in monoclonal antibodies are broken in a reducing environment, leading to the degradation of antibody monomers into inactive byproducts such as half antibodies and separate heavy chains and/or light chains. While antibody reduction is a risk for all processes, different antibodies have different susceptibilities to reduction due to the number and structure of disulfide bonds.

As cell culture processes progress and cells die, enzymes from the thioredoxin and glutathione reductase pathways are released and lead to an increasingly reductive environment in the cell culture fluid which leads to antibody reduction. This reduction can occur at multiple stages in the cell culture process; namely in the bioreactor itself, during clarification, in the harvest hold vessel, and even during protein A processing. The harvest hold vessel represents the largest risk of reduction, especially if cells are lysed during clarification. While antibody reduction is a risk for all monoclonal antibody processes, it has been shown that different antibodies can demonstrate vastly differing susceptibilities to reduction due to the number and structure of disulfide bonds present. These susceptibilities can be broadly characterized by antibody subtype (IgG1, IgG2 etc.) and by light chain subtype (kappa and lambda), although differences in reduction susceptibility are also present within these broad classes. Some methods such as chemical reduction assays, while useful for predicting which antibodies will have issues in production and process development, cannot be used to assess different culture conditions as it only takes purified antibodies and subjects them to a chemical reducing reagent, which is a stark difference to the enzyme induced reduction and complex environment present in the cell culture process.

Therefore, there is a need in the art for a method of determining protein reduction susceptibility that mimics the actual culturing environment for quick and accurate results.

### SUMMARY

Provided herein are small scale assays that can be used to accurately predict the risk of antibody reduction for different proteins (e.g., monoclonal antibodies) during the cell culture process, including bench scale assays. The provided assays can be used to modify manufacturing processes to prevent or reduce the level of reduction of proteins. Applicant unexpectedly discovered that the performance of the assays is optimized by using glass vials having an interior volume of about 1 mL to about 3 mL (e.g., about 2.5 mL) that have less than 100 microliters of headspace.

Accordingly, in one aspect, provided herein is a method of determining the reduction susceptibility of a protein, comprising: (a) providing a first cell culture fluid and a second cell culture fluid, wherein the first cell culture fluid and the second cell culture fluid are identical, except the first cell culture fluid comprises a cell lysate and the second cell culture fluid is essentially free of the cell lysate; (b) mixing the first cell culture fluid and the second cell culture fluid at a series of ratios, thereby preparing a set of samples; (c) adding the protein to each sample of the set of samples and incubating each sample of the set of samples in an air-sealed container (i) having an interior volume of about 1 mL to about 3 mL, and (ii) having less than 100 microliters of headspace; (d) determining the level of protein reduction and/or the redox potential in each sample of the set of samples; and (e) determining the reduction susceptibility of the protein based on the level of protein reduction and/or the redox potential determined for each sample of the set of samples in step (d), thereby determining the reduction susceptibility of the protein.

In some embodiments, the cell lysate was obtained through homogenization of cells.

In some embodiments, the cell lysate was centrifuged to remove cellular debris before step (b).

In some embodiments, the second cell culture fluid is obtained by removing essentially all of the cells from a cell culture medium.

In some embodiments, the protein is a recombinant protein.

In some embodiments, the recombinant protein is an antibody, or antigen-binding fragment thereof.

In some embodiments, the antibody is a monoclonal antibody.

In some embodiments, the level of protein reduction is determined by assessing the oxidation state of the disulfide bonds in the protein.

In some embodiments, the redox potential is determined by measuring one or more of lactate dehydrogenase (LDH), the nicotinamide adenine dinucleotide (NAD+)/reduced NAD+(NADH) redox couple, the NADP+/reduced NADP+(NADPH) redox couple, NADPH, thioredoxin (Trx), and/or glutathione (Grx).

In some embodiments, the redox potential is determined by using a redox potential probe.

In some embodiments, the level of protein reduction is determined by the amount of intact and/or reduced protein after the incubating with each sample of the set of samples.

In some embodiments, the amount of intact and/or reduced protein is determined using a native SDS-PAGE gel.

In some embodiments, the amount of intact and/or reduced protein is determined using a microfluidic device.

In some embodiments, the method described herein further comprises determining the level of protein reduction and the redox potential in each sample of the set of samples and further comprising generating a graph of the protein reduction susceptibility based on the determined redox potential and the determined level of intact or reduced protein for each sample in the set of samples.

In some embodiments, the air-sealed container is an air-sealed glass vial.

In some embodiments, the air-sealed container has an interior volume of about 2 mL to about 2.5 mL.

In some embodiments, the air-sealed container has less than 50 microliters, less than 10 microliters, or 0 microliter of headspace.

In some embodiments, the protein is incubated with each sample of the set of samples for about 0.5 to about 4 hours prior to step (c).

In some embodiments, the method described herein further comprises comparing the reduction susceptibility of the protein with a reference standard.

In some embodiments, the reference standard is a standard curve of reduction susceptibility of a reference protein.

In some embodiments, the method described herein further comprises selecting the protein for further development based on the reduction susceptibility of the protein.

In another aspect, provided herein is a method of modifying a culturing condition or cell culture fluid bioprocessing condition of a cell culture medium used for the production of a protein, comprising: (a) providing a first cell culture fluid and a second cell culture fluid, wherein the first cell culture fluid and the second cell culture fluid are identical, except the first cell culture fluid comprises a cell lysate and the second cell culture fluid is essentially free of the cell lysate; (b) mixing the first cell culture fluid and the second cell culture fluid at a series of ratios, thereby preparing a set of samples; (c) adding the protein to each sample of the set of samples and incubating each sample of the set of samples in an air-sealed container (i) having an interior volume of about 1 mL to about 3 mL, and (ii) having less than 100 microliters of headspace; (d) determining the level of protein reduction and/or the redox potential in each sample of the set of samples; (e) determining the reduction susceptibility of the protein based on the level of protein reduction and/or the redox potential determined for each sample of the set of samples in step (d); and (f) modifying one or more culturing conditions or cell culture fluid bioprocessing conditions of the cell culture medium based on the reduction susceptibility of the protein, thereby modifying the culturing condition or the cell culture fluid bioprocessing condition used for the production of the protein.

In some embodiments, the cell lysate was obtained through homogenization of cells.

In some embodiments, the cell lysate was centrifuged to remove cellular debris before step (b).

In some embodiments, the second cell culture fluid is obtained by removing essentially all of the cells from a cell culture medium.

In some embodiments, the protein is a recombinant protein.

In some embodiments, the recombinant protein is an antibody, or antigen-binding fragment thereof.

In some embodiments, the antibody is a monoclonal antibody.

In some embodiments, the level of protein reduction is determined by assessing the oxidation state of the disulfide bonds in the protein.

In some embodiments, the redox potential is determined by measuring one or more of lactate dehydrogenase (LDH), the nicotinamide adenine dinucleotide (NAD+)/reduced NAD+(NADH) redox couple, the NADP+/reduced NADP+(NADPH) redox couple, NADPH, thioredoxin (Trx), and/or glutathione (Grx).

In some embodiments, the redox potential is determined by using a redox potential probe.

In some embodiments, the level of protein reduction is determined by the amount of intact and/or reduced protein after incubating with each sample of the set of samples.

In some embodiments, the amount of intact and/or reduced protein is determined using a native SDS-PAGE gel.

In some embodiments, the amount of intact and/or reduced protein is determined using a microfluidic device.

In some embodiments, the air-sealed container is an air-sealed glass vial.

In some embodiments, the air-sealed container has an interior volume of about 2 mL to about 2.5 mL.

In some embodiments, the air-sealed container has less than 50 microliters, less than 10 microliters, or 0 microliter of headspace.

In some embodiments, the protein is incubated with each sample of the set of samples for about 0.5 to about 4 hours prior to step (c).

In some embodiments, the modifying of the one or more culturing conditions or the cell culture fluid bioprocessing conditions of the cell culture medium comprises adjusting one or more: a component of a liquid culture medium composition, pH, temperature, and dissolved oxygen during the production of the protein.

In another aspect, provided herein is a method of generating a standard curve of reduction susceptibility of a protein, comprising: (a) providing a first cell culture fluid and a second cell culture fluid, wherein the first cell culture fluid and the second cell culture fluid are identical, except the first cell culture fluid comprises a cell lysate and the second cell culture fluid is essentially free of the cell lysate; (b) mixing the first cell culture fluid and the second cell culture fluid at a series of ratios, thereby preparing a set of samples; (c) adding the protein to each sample of the set of samples and incubating each sample of the set of samples in an air-sealed container (i) having an interior volume of about 1 mL to about 3 mL, and (ii) having less than 100 microliters of headspace; (d) determining the level of protein reduction and the redox potential in each sample of the set of samples; and (e) generating a standard curve of reduction susceptibility of the protein based on the determined level of redox potential and the determined level of protein reduction for each sample of the set of samples, thereby generating the standard curve of reduction susceptibility of the protein.

In some embodiments, the cell lysate was obtained through homogenization of cells.

In some embodiments, the cell lysate was centrifuged to remove cellular debris before step (b).

In some embodiments, the second cell culture fluid is obtained by removing essentially all of the cells from a cell culture medium.

In some embodiments, the protein is a recombinant protein.

In some embodiments, the recombinant protein is an antibody, or antigen-binding fragment thereof.

In some embodiments, the antibody is a monoclonal antibody.

In some embodiments, the level of protein reduction is determined by assessing the oxidation state of the disulfide bonds in the protein.

In some embodiments, the redox potential is determined by measuring one or more of lactate dehydrogenase (LDH), the nicotinamide adenine dinucleotide (NAD+)/reduced NAD+(NADH) redox couple, the NADP+/reduced NADP+(NADPH) redox couple, NADPH, thioredoxin (Trx), and/or glutathione (Grx).

In some embodiments, the redox potential is determined by using a redox potential probe.

In some embodiments, the level of protein reduction is determined by the amount of intact and/or reduced protein after incubating with each sample of the set of samples.

In some embodiments, the amount of intact and/or reduced protein is determined using a native SDS-PAGE gel.

In some embodiments, the amount of intact and/or reduced protein is determined using a microfluidic device.

In some embodiments, the air-sealed container is an air-sealed glass vial.

In some embodiments, the air-sealed container has an interior volume of about 2 mL to about 2.5 mL.

In some embodiments, the air-sealed container has less than 50 microliters, less than 10 microliters, or 0 microliter of headspace.

In some embodiments, the protein is incubated with each sample of the set of samples for about 0.5 to about 4 hours prior to step (c).

In another aspect, provided herein is a method of determining the reduction susceptibility of a protein, comprising: (a) adding the protein to each sample of a set of samples prepared by mixing a first cell culture fluid and a second cell culture fluid at a series of ratios, wherein the first cell culture fluid and the second cell culture fluid are identical, except the first cell culture fluid comprises a cell lysate and the second cell culture fluid is essentially free of the cell lysates; (b) incubating each sample of the set of samples in an air-sealed container (i) having an interior volume of about 1 mL to about 3 mL, and (ii) having less than 100 microliters of headspace; and (c) determining the level of protein reduction and/or the redox potential in each sample of the set of samples to determine the reduction susceptibility of the protein based on the level of protein reduction and/or the redox potential, thereby determining the reduction susceptibility of the protein.

In some embodiments, the cell lysate was obtained through homogenization of cells.

In some embodiments, the cell lysate was centrifuged to remove cellular debris before step (b).

In some embodiments, the second cell culture fluid is obtained by removing essentially all of the cells from a cell culture medium.

In some embodiments, the protein is a recombinant protein.

In some embodiments, the recombinant protein is an antibody, or antigen-binding fragment thereof.

In some embodiments, the antibody is a monoclonal antibody.

In some embodiments, the level of protein reduction is determined by assessing the oxidation state of the disulfide bonds in the protein.

In some embodiments, the redox potential is determined by measuring one or more of lactate dehydrogenase (LDH), the nicotinamide adenine dinucleotide (NAD+)/reduced NAD+(NADH) redox couple, the NADP+/reduced NADP+(NADPH) redox couple, NADPH, thioredoxin (Trx), and/or glutathione (Grx).

In some embodiments, the redox potential is determined by using a redox potential probe.

In some embodiments, the level of protein reduction is determined by the amount of intact and/or reduced protein after incubating with each sample of the set of samples.

In some embodiments, the amount of intact and/or reduced protein is determined using a native SDS-PAGE gel.

In some embodiments, the amount of intact and/or reduced protein is determined using a microfluidic device.

In some embodiments, the methods described herein further comprises determining the level of protein reduction and the redox potential in each sample of the set of samples and further comprising generating a graph of the protein reduction susceptibility based on the determined redox potential and the determined level of intact or reduced protein for each sample in the set of samples.

In some embodiments, the air-sealed container is an air-sealed glass vial.

In some embodiments, the air-sealed container has an interior volume of about 2 mL to about 2.5 mL.

In some embodiments, the air-sealed container has less than 50 microliters, less than 10 microliters, or 0 microliter of headspace.

In some embodiments, the protein is incubated with each sample of the set of samples for about 0.5 to about 4 hours prior to step (c).

In some embodiments, the method described herein further comprises comparing the reduction susceptibility of the protein with a reference standard.

In some embodiments, the reference standard is a standard curve of reduction susceptibility of a reference protein.

In some embodiments, the method described herein further comprises selecting the protein for further development based on the reduction susceptibility of the protein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic illustration showing the reduction of protein disulfide bond.

FIG. 2 is a schematic illustration of antibody reduction during cell culture development processes.

FIG. 3 is a schematic illustration of an experimental outline of an example method for determining the correlation between reduction markers.

FIGS. 4A-4B show the correlation between the level of LDH and ORP in the series dilution of the cell culture medium.

FIG. 5 is a schematic illustration of an experimental outline of an example method for determining protein reduction susceptibility described herein. Specifically, 7-day CHO culture is harvested and partially lysed by pressure homogenization. Lysed and unlysed culture supernatants are mixed to achieve specific lactate dehydrogenase (LDH) values. Antibody is added and incubated at a specific temperature for a specific period of time. Samples are processed for reduction analysis by non-reducing SDS gel.

FIG. 6A shows the effect of using different containers when performing an example method for determining protein reduction susceptibility described herein. Different containers were tested in a time-based protein reduction assay to show the impact of permeability of air into the different containers over time.

FIG. 6B shows the results of an example method for determining protein reduction susceptibility described herein. The protein reduction assay was performed using different antibody subtypes using fully lysed cultures and sampling at different time intervals.

FIG. 6C shows the reduction susceptibility of different types of protein (subtypes of IgG) measured by the levels of LDH in the cell culture medium dilutions.

FIG. 7 shows the reduction susceptibility of different types of protein (subtypes of IgG) measured by the levels of ORP in the cell culture medium dilutions.

### DETAILED DESCRIPTION

The present disclosure relates to methods of determining the reduction susceptibility of a protein and methods of adjusting the culturing condition or methods of manufacturing of the protein based on the determined reduction susceptibility.

While chemical reduction assays can be used to detect antibody reduction, they do not mimic the process environment and cannot be used to assess process conditions for mitigation of reduction risk. In the present disclosure, a more process relevant reduction assay in a cell culture environment is described. One of the advantages of a cell culture-based assay is the ability to probe process conditions, such as oxidation reduction potential (ORP) for their effects on a protein's susceptibility to reduction.

The small-scale assay described herein uses cell culture fluid in a more relevant environment to assess changes in cell culture parameters effects on reduction susceptibility. In this assay, cell lysate is mixed at different ratios with clarified culture fluid to mimic different culture environments in small, sealed containers. In this way, the reducing environment of the bioreactor, clarification, and harvest hold vessels of a manufacturing process can be simulated at the bench scale.

This assay was demonstrated to show changing susceptibilities to reduction based on changing cell culture parameters known to influence reduction including the amount of cell lysis present, temperature, and oxygen content. This assay is able to accurately determine the reduction susceptibility of a protein. This assay was further coupled with oxidation-reduction potential (ORP) measurements to correlate with larger scale findings, demonstrating changing reduction patterns as the ORP within the culture fluid is changing. This assay is also adaptable to add to any process production runs and test harvest hold conditions following cell culture production and clarification as needed. These assays can also be used to study reduction mechanics and prevention techniques including changing process parameters and cell culture additives to prevent protein reduction in large-scale processes.

### Definitions

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell culture medium, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition functionally inert, but at a low concentration. Similar meaning can be applied to the term “absence of,” where referring to the absence of a particular substance or its source thereof of a composition.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “subject” refers to any animal, preferably a human patient, livestock, or other domesticated animal.

“Culture” or “cell culture” refers to the maintenance, growth and/or differentiation of cells (e.g., mammalian cells) in an in vitro environment. Cell culture can refer in some embodiments to batch cell culture, fed-batch cell culture, or perfusion cell culture. “Cell culture media,” “culture media” (singular “medium” in each case), “supplement” and “media supplement” refer to nutritive compositions that cultivate cell cultures.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., multi-specific antibodies, bi-specific antibodies, single-chain antibodies, diabodies, and linear antibodies formed from these antibodies or antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain, a variable domain of light chain or a VHH). Non-limiting examples of antibody fragments include, e.g., Fab, Fab′, F(ab′)2, and Fv fragments, scFv, and VHH.

The term “recombinant protein”, as used herein, refers to any protein or biologically active portion thereof (for example, a portion that retains biological activity of the full protein) that is not a reporter or marker gene (for example, a green fluorescent protein) expressed from recombinant genetic material encoding amino acids, including peptides, polypeptides, proteins, oligoproteins and/or fusion proteins. A recombinant protein product may include a therapeutic, prophylactic, or diagnostic product.

The term “production of protein” includes techniques used to grow cells, e.g., recombinant cells, in culture and to obtain a protein of interest produced by the cultured cells in an appropriate form for use. The manufacturing process can include various steps, including, but not limited to one or more of the following: inserting of a gene of interest into a host cell to create an engineered host cell, culturing the host cell to expand the number of cells, inducing expression of the protein of interest by the host cell, screening for host cells expressing the protein of interest, harvesting the protein of interest, e.g., by separating the protein of interest from the cultured cells and cell culture medium, and/or purifying the protein of interest. The protein of interest can be an endogenous protein expressed by the native cell, or a recombinant heterologous protein encoded in an expression vector inserted into the cell (either transiently or stably).

As used herein, a “cell culture fluid” is a fluid obtained from a cell culture, e.g., a fed-batch culture, batch culture, or perfusion culture. The cell culture fluid can contain the cell culture medium, supplements added to the medium during cell culture, cellular components, and/or metabolites during released from the cells. In some embodiments, a cell culture fluid contains lysed cells or cell lysate. In some embodiments, a cell culture fluid is essentially free of lysed cells or cell lysate.

As used herein the term “disulfide bond” or “disulfide bridge” or “S—S bond” includes the covalent bond formed between two sulfur atoms. The amino acid cysteine comprises a thiol group that can form a disulfide bond or bridge with a second thiol group. The second thiol group can be found in the side chain of a residue on the same polypeptide or protein (intra-disulfide bond), or on a different polypeptide or protein (inter-disulfide bond). Such bonds are created during protein biosynthesis and/or by oxidation of sulfhydryl groups, a process referred to as oxidative protein folding. Exemplary methods for detecting disruption of a disulfide bond include non-reducing SDS-PAGE and native 2-dimensional electrophoresis. Additional methods for detecting disruption of a disulfide bond are known in the art.

The terms “disulfide bond-containing protein” and “disulfide bond-containing protein” are used interchangeably herein and refer to a protein that in its properly folded state contains one or more disulfide bonds. The disulfide bond-containing protein can have a disulfide bond that is inter- or intra-molecular. Such inter- or intra-molecular disulfide bonds are present in a properly folded disulfide bond-containing protein. The activity of the disulfide bond-containing protein can be dependent on the presence and reduction state of the disulfide bonds. Many molecules function best with the formation of stable inter- and intra-molecular disulfide bonds that contribute to proper folding, such as immunoglobulins and cell surface receptors containing immunoglobulin domains, ribonucleases, lactalbumin, insulin, keratin, hemagglutinin, viral membrane proteins, neuroendocrine protein 7B2, epidermal growth factor (EGF), androgenic gland hormone, sulfide dehydrogenase, and lysozyme. Many therapeutic proteins, such as antibodies, EGF, and insulin, contain disulfide bonds. Manufacturing a therapeutic protein of interest under conditions that increase disulfide bond reduction can result in low yield of the intact protein of interest. As stated herein, term “yield” refers to the amount of protein of interest obtained that is intact, active, properly folded, and with the correct pairing of disulfide bonds. A process may produce a high overall amount of protein of interest, but if significant reduction of the disulfide bonds occurs in the protein of interest, the yield of intact protein may be low.

The term “reduced protein” means a protein that is exposed to reducing conditions sufficient to reduce a reducible residue in the protein structure, such as a cysteine. If the reduced protein contains a thiol group, or sulfur-containing residue, then the thiol group in the reduced protein exists in a state in which it is reduced. For instance, a reduced protein that contains a cysteine residue will exist in a state in which the sulfur atom of the cysteine residue is in the reduced state, often indicated as “—SH.” A reduced protein can be a disulfide bond-containing protein. A disulfide bond-containing protein can become a reduced protein by exposure to reducing conditions that cause one or more disulfide bonds (disulfide bridges) in the disulfide bond-containing protein to break, which can contribute to destabilization of the disulfide bond-containing protein and potential loss of activity or function of the disulfide bond-containing protein.

The term “redox potential” or “oxidation reduction potential (ORP),” also known as REDOX, is a measurement that reflects the ability of a molecule to oxidize or reduce another molecule. Oxidation is the loss of electrons, so oxidizers accept electrons from other molecules. Reduction is the gain of electrons, so reducers donate electrons to other molecules. Oxidation reduction potential is measured as a single voltage in millivolts (mV). Oxidizers have a positive ORP value, while reducers have a negative ORP value. Redox potential characterizes the free energy cost and direction of reactions involving electron transfer, one of the most ubiquitous and important of biochemical reactions. Such reduction-oxidation reactions are characterized by a free energy change that shares some conceptual features with that used to describe pKa in acid-base reactions where proton transfer is involved rather than electron transfer. The redox potential can be used as a measure of the driving force for a given oxidation-reduction reaction of interest. In some embodiments, the redox potential characterizes the oxidizing/reducing power of a sample, e.g., a mixture of cell culture fluid. Exemplary methods that can be used for determining the redox potential are described herein. Additional methods for determining the redox potential are known in the art.

The term “thioredoxin system” means the enzymes thioredoxin reductase-1 (TrxR1), and thioredoxin-1 (Trx-1), and the cofactor NADPH. These three components make up the thioredoxin system which supports several processes needed for eukaryotic cell function including cell proliferation, antioxidant defense, and redox signaling (Lu et al., 2014, Free Radic. Biol. Med., 66:75-87).

The term “glutathione system” means the components glutathione, glutathione reductase (GR), glutaredoxin (Grx), and the cofactor NADPH (Lillig et al., 2008, Biochim. Biophys. Acta—Gen. Subj., 1780:1304-1317).

The glutathione system and the thioredoxin system are collectively and alternatively referred to herein as “reductase system” or “reductase systems.” That is, the term “reductase systems” encompasses both the glutathione system and/or the thioredoxin system.

As used herein, “developability” refers to the feasibility of proteins, e.g., disulfide bond-containing proteins, to successfully progress from discovery to development via evaluation of their physicochemical properties, e.g., their reduction susceptibility. Other properties include the tendency for self-interaction and aggregation, thermal stability, colloidal stability, and optimization of their properties through sequence engineering. Selection of a protein with desired properties based on biological function, efficacy, safety, and developability allows for a streamlined and successful downstream production. In some embodiments, the method described herein includes evaluating the developability of a protein based on its reduction susceptibility. In some embodiments, the method described herein includes selecting a protein for further development based on its developability.

### Methods of Determining Protein Reduction Susceptibility

In one aspect, provided herein is a method of determining the reduction susceptibility of a protein, comprising: (a) providing a first cell culture fluid and a second cell culture fluid, wherein the first cell culture fluid and the second cell culture fluid are identical, except the first cell culture fluid comprises a cell lysate and the second cell culture fluid is essentially free of the cell lysate; (b) mixing the first cell culture fluid and the second cell culture fluid at a series of ratios, thereby preparing a set of samples; (c) adding the protein to each sample of the set of samples and incubating each sample of the set of samples in an air-sealed container (i) having an interior volume of about 1 mL to about 3 mL, and (ii) having less than 100 microliters of headspace; (d) determining the level of protein reduction and/or the redox potential in each sample of the set of samples; and (e) determining the reduction susceptibility of the protein based on the level of protein reduction and the redox potential determined for each sample of the set of samples in step (d), thereby determining the reduction susceptibility of the protein.

In another aspect, provided herein is a method of determining the reduction susceptibility of a protein, comprising: (a) adding the protein to each sample of a set of samples prepared by mixing a first cell culture fluid and a second cell culture fluid at a series of ratios, wherein the first cell culture fluid and the second cell culture fluid are identical, except the first cell culture fluid comprises a cell lysate and the second cell culture fluid is essentially free of the cell lysates; (b) incubating each sample of the set of samples in an air-sealed container (i) having an interior volume of about 1 mL to about 3 mL, and (ii) having less than 100 microliters of headspace; and (c) determining the level of protein reduction and/or the redox potential in each sample of the set of samples to determine the reduction susceptibility of the protein based on the level of protein reduction and the redox potential, thereby determining the reduction susceptibility of the protein.

The cell lysate in the first cell culture fluid can be obtained through any suitable method known in the art. The lysis of the cell can be achieved using physical methods or reagent-based methods. In some embodiments, the cell lysate was obtained through homogenization of cells. In some embodiments, the cell lysate was obtained through enzymatic treatment. In some embodiments, the cell lysate was centrifuged to remove cellular debris before incubating the protein in each sample of the set of samples in the air-sealed container.

In some embodiments, the cell lysate in the first cell culture fluid includes one or more enzymes, cell organelles and/or metabolites.

In some embodiments, the cell lysate includes one or more enzymes that contribute to the reduction of the protein. In some embodiments, the cell lysate includes one or enzymes from the thioredoxin and glutathione reductase pathways.

In some embodiments, the second cell culture fluid is obtained by removing essentially all of the cells from a cell culture medium.

The first and second cell culture fluid are mixed at a series of ratios to create a set of samples for the determination of the protein reduction susceptibility. In some embodiments, the ratios between the first and second cell culture fluid in the set of samples increase or decrease by a 2-fold, 3-fold, 4-fold, 5-fold, 10-fold, 33-fold, or 100-fold interval. In some embodiments, the first and second cell culture fluid are mixed at a ratio of about 1:10⁵, 1:10⁴, 1:1000, 1:100, 1:10, 1:1, 10:1, 100:1, 1000:1, 10⁴:1 or any other suitable ratios. The set of samples created by mixing the first and second cell culture fluid provides a ladder of reducing environment for the testing of the reduction susceptibility of the protein. In some embodiments, the set of samples further include one or more reference samples that have only the first cell culture fluid or only the second cell culture fluid.

The protein in the methods described herein can be any suitable protein. In some embodiments, the protein is a recombinant protein.

In some embodiments, the recombinant protein is an antibody, or antigen-binding fragment thereof. In some embodiments, the antibody is a monoclonal antibody.

In some embodiments, the protein is an immunoglobulin. As used herein, the term “immunoglobulin” comprises various broad classes of polypeptides or proteins that can be distinguished biochemically. Those skilled in the art will appreciate that immunoglobulin heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgG, or IgE, respectively. The immunoglobulin subclasses (subtypes) e.g., IgG1, IgG2, IgG3, IgG4, IgA1, IgA2, etc. are well characterized and are known to confer functional specialization. Modified versions of each of these immunoglobulins are readily discernible to the skilled artisan in view of the instant disclosure and, accordingly, are within the scope of this disclosure.

In some embodiments, the reduction susceptibility is determined for a disulfide bond-containing protein. In some embodiments, the level of protein reduction is determined by assessing the oxidation state of the disulfide bonds in the protein. The oxidation state of the disulfide bond.

In some embodiments, the redox potential is determined by measuring the level of one or more molecules that are involved in the oxidation reduction reactions in cells and during protein reduction. These molecules include, but are not limited to, lactate dehydrogenase (LDH), the nicotinamide adenine dinucleotide (NAD+)/reduced NAD+(NADH) redox couple, the NADP+/reduced NADP+(NADPH) redox couple, NADPH, thioredoxin (Trx), and glutathione (Grx). In some embodiments, the redox potential is determined by measuring the level of one or more molecules that are involved in the glutathione system. In some embodiments, the redox potential is determined by measuring the level of one or more molecules that are involved in the thioredoxin system.

Methods of detecting and/or determining the level of the molecules for redox potential are known in the art. For example, the level of LDH can be measured by the CEDEX bioanalyzer (Roche Diagnostics CEDEX BIO LDH, Roche Diagnostics 06343767001). The level of thioredoxin can be measured by the Thioredoxin Reductase Colorimetric Assay Kit (Cayman Chemical, Item No. 10007892). The level of glutathione can be measured by the Glutathione Colorimetric Detection Kit (Thermo Fisher, Catalog number: EIAGSHC). The level of NADPH can be measured by the NADPH Assay Kit (Colorimetric) (Abcam, ab186031). The level of the nicotinamide adenine dinucleotide (NAD+)/reduced NAD+(NADH) redox couple, and/or the NADP+/reduced NADP+(NADPH) redox couple can be measured by the NAD/NADH-Glo™ and NADP/NADPH-Glo™ Assays (Promega). Other commercially available methods and kits for determining redox potential are known in the art.

In some embodiments, the redox potential is determined by using a redox potential probe. The redox potential probe measures the ability of a solution to act as an oxidizing or reducing agent. The ionic potential information an ORP probe obtains is thereby used to indicate the redox potential of a sample, e.g., a mixture of the first and second cell culture fluid in the method described herein. Probes that are used to determine and detecting the redox potential are commercially available and known in the art. For example, the redox potential (level of ORP) can be determined using a laboratory ORP sensor (METTLER TOLEDO).

In some embodiments, the level of protein reduction is determined by the amount of intact and/or reduced protein after incubating with each sample of the set of samples. Methods of quantifying the level of reduced proteins and the level of intact protein are known in the art.

As used herein, “intact protein” refers to a properly folded protein without any disulfide bond reduction. “Reduced protein” refers to a protein with at least 0.1%, 1%, 5%, 10%, 15%, 20%, 25%, 30% or more of disulfide bond reduction.

In some embodiments, the amount of intact and/or reduced protein is determined using a native, non-reducing SDS-PAGE gel. In some embodiments, the amount of intact and/or reduced protein is determined using a microfluidic device. Any other suitable methods can be used herein to determine the amount of intact and/or reduced protein.

In some embodiments, the method described herein further comprises generating a graph of the protein reduction susceptibility based on the determined redox potential and the determined level of intact or reduced protein for each sample in the set of samples.

In some embodiments, the graph establishes the correlation between the amount of intact and/or reduced protein with the redox potential of each sample in the set of samples. In some embodiments, the graph establishes the correlation between the protein reduction susceptibility with the redox potential of each sample in the set of samples.

The container used in the methods described herein are critical in determining the protein reduction susceptibility. Specifically, the disclosure provides a small-scale assay that can be performed in a high-throughput manner. Therefore, the interior volume of the containers in the methods described herein should be within a certain range. In some embodiments, the interior volume of the container is about 0.1 mL to about 100 mL, about 1 mL to about 100 mL, about 1 mL to about 10 mL, or 1 mL to about 3 mL. In some embodiments, interior volume of the container is about 1 mL, about 2 mL, about 3 mL, about 4 mL, about 5 mL, about 6 mL, about 7 mL, about 8 mL, about 9 mL, or about 10 mL. In some embodiments, interior volume of the container is about 2 mL. In some embodiments, interior volume of the container is about 2 mL to about 2.5 mL. In some embodiments, interior volume of the container is about 2.5 mL.

The material of the container is also critical in achieving the desired result of the method described herein. Specifically, the container is air-sealed to prevent the oxidation of the protein and accurate assessment of protein reduction. Therefore, the material of the container should be minimally air-permeable. In some embodiments, the air-sealed container is an air-sealed glass vial. In some embodiments, the air-sealed container is an air-sealed metal container. In some embodiments, the air-sealed container is an air-sealed plastic container.

In the method described herein, the first and second cell culture fluid is first mixed at different ratios, and the protein to be tested is added to each mixture and incubated. To prevent oxidation reaction of the protein and accurately assess protein reduction, there should be minimum headspace between the fluid mixture and the sealer, e.g. the cap of the glass vial. The mixing of the first and second cell culture fluid and/or the mixing of the protein with the cell culture fluid samples can be performed using any suitable methods. For example, the mixing is performed by inverting the container (e.g., glass vial) several times. The mixing can also be performed by gently pipetting the liquid in the container. In some embodiments, the mixing is performed using a method that does not disrupt the protein structure in the mixture.

As used herein, the term “headspace” is the distance or volume from the top of the container to the top of the liquid (e.g., the sample including the mixture of the first and second cell culture fluid). In some embodiments, when making this measurement, any extensions of the cover or lid above the body of the container are disregarded. In some embodiments, the air-sealed container has less than 200 microliters, less than 150 microliters, less than 100 microliters, less than 50 microliters, less than 10 microliters, or 0 microliter of headspace.

In some embodiments, the protein is incubated with each sample of the set of samples for minutes, hours or days. In some embodiments, the protein is incubated with each sample of the set of samples for about 0.5 to about 24 hours, about 0.5 to about 12 hours, about 0.5 to about 6 hours, about 0.5 to about 5 hours, about 0.5 to about 4 hours, about 0.5 to about 3 hours, about 0.5 to about 2 hours, or about 0.5 to about 1 hour, prior to adding the protein to each sample of the set of samples and incubating each sample of the set of samples in an air-sealed container.

In some embodiments, the protein is incubated with each sample of the set of samples for about 24 hours to about 36 hours, about 24 hours to about 48 hours, about 24 hours to about 60 hours, about 24 hours to about 72 hours, about 1 day to about 4 days, about 1 day to about 5 days, about 1 day to about 6 days, about 1 day to about 7 days, about 1 day to about 8 days, about 1 day to about 9 days, about 1 day to about 10 days or more, prior to adding the protein to each sample of the set of samples and incubating each sample of the set of samples in an air-sealed container.

The incubation of the protein with each sample of the set of samples can be performed under any suitable conditions. In some embodiments, the incubation is performed at a temperature of about 37° C. In some embodiments, the incubation is performed at room temperature. In some embodiments, the incubation is performed at a temperature of about 4° C.

In some embodiments, the incubation is performed under a CO2 level of about 5%. In some embodiments, the incubation is performed under 95% Relative Humidity. In some embodiments, the incubation is performed under a physiological pH condition (e.g., about 7.3 to about 7.45 pH value). In some embodiments, the incubation is performed under a condition that would mimic the cell culture and the holding tank conditions. In some embodiment, the temperature for the holding tank is lower than the temperature of the reactor. In some embodiments, the temperature for the holding tank is room temperature. In some embodiments, the temperature for the holding tank is about 4° C. to about 10° C. In some embodiments, the temperature for the holding tank is about 4° C.

In some embodiments, the method described herein further comprises comparing the reduction susceptibility of the protein with a reference standard.

In some embodiments, the reference standard is a standard curve of reduction susceptibility of a reference protein. The reference protein can be, for example, a standard protein whose reduction susceptibility has been previous determined to be acceptable for further development.

In some embodiments, the method described herein further comprises selecting the protein for further development based on the reduction susceptibility of the protein. In some embodiments, the method described herein includes evaluating the developability of a protein based on its reduction susceptibility. In some embodiments, the method described herein includes selecting a protein for further development based on its developability.

### Method of Modifying Cell Culture Fluid Bioprocessing Conditions

In another aspect, provided herein is a method of modifying a culturing condition or cell culture fluid bioprocessing condition of a cell culture medium used for the production of a protein, comprising: (a) providing a first cell culture fluid and a second cell culture fluid, wherein the first cell culture fluid and the second cell culture fluid are identical, except the first cell culture fluid comprises a cell lysate and the second cell culture fluid is essentially free of the cell lysate; (b) mixing the first cell culture fluid and the second cell culture fluid at a series of ratios, thereby preparing a set of samples; (c) adding the protein to each sample of the set of samples and incubating each sample of the set of samples in an air-sealed container (i) having an interior volume of about 1 mL to about 3 mL, and (ii) having less than 100 microliters of headspace; (d) determining the level of protein reduction and/or the redox potential in each sample of the set of samples; (e) determining the reduction susceptibility of the protein based on the level of protein reduction and the redox potential determined for each sample of the set of samples in step (d); and (f) modifying one or more culturing conditions or cell culture fluid bioprocessing conditions of the cell culture medium based on the reduction susceptibility of the protein, thereby modifying the culturing condition or the cell culture fluid bioprocessing condition used for the production of the protein.

In another aspect, provided herein is a method of modifying a culturing condition or cell culture fluid bioprocessing condition of a cell culture medium used for the production of a protein, comprising: (a) adding the protein to each sample of a set of samples prepared by mixing a first cell culture fluid and a second cell culture fluid at a series of ratios, wherein the first cell culture fluid and the second cell culture fluid are identical, except the first cell culture fluid comprises a cell lysate and the second cell culture fluid is essentially free of the cell lysates; (b) incubating each sample of the set of samples in an air-sealed container (i) having an interior volume of about 1 mL to about 3 mL, and (ii) having less than 100 microliters of headspace; (c) determining the level of protein reduction and/or the redox potential in each sample of the set of samples to determine the reduction susceptibility of the protein based on the level of protein reduction and the redox potential; and (d) modifying one or more culturing conditions or cell culture fluid bioprocessing conditions of the cell culture medium based on the reduction susceptibility of the protein, thereby modifying the culturing condition or the cell culture fluid bioprocessing condition used for the production of the protein.

In some embodiments, the modifying of the one or more culturing conditions or the cell culture fluid bioprocessing conditions of the cell culture medium comprises adjusting one or more: a component of a liquid culture medium composition, pH, temperature, and dissolved oxygen during the production of the protein.

In some embodiments, the modifying of the one or more culturing conditions or cell culture fluid bioprocessing conditions of the cell culture medium includes adjusting one or more threshold parameters in the cell culture medium. These threshold parameters include, without limitation, optical density (OD), dissolved oxygen (DO), pH, concentration of nutrient in the culture medium, total concentration of the first carbon source added to the culture medium, or any combination thereof.

In some embodiments, the modifying of the one or more culturing conditions or cell culture fluid bioprocessing conditions of the cell culture medium includes filtering out the reducing agents (byproducts of reduction reactions) from the cell culture medium.

### Method of Generating Standard Curves

In another aspect, provided herein is a method of generating a standard curve of reduction susceptibility of a protein, comprising: (a) providing a first cell culture fluid and a second cell culture fluid, wherein the first cell culture fluid and the second cell culture fluid are identical, except the first cell culture fluid comprises a cell lysate and the second cell culture fluid is essentially free of the cell lysate; (b) mixing the first cell culture fluid and the second cell culture fluid at a series of ratios, thereby preparing a set of samples; (c) adding the protein to each sample of the set of samples and incubating each sample of the set of samples in an air-sealed container (i) having an interior volume of about 1 mL to about 3 mL, and (ii) having less than 100 microliters of headspace; (d) determining the level of protein reduction and the redox potential in each sample of the set of samples; and (e) generating a standard curve of reduction susceptibility of the protein based on the determined level of redox potential and the determined level of protein reduction for each sample of the set of samples, thereby generating the standard curve of reduction susceptibility of the protein.

In another aspect, provided herein is a method of generating a standard curve of reduction susceptibility of a protein, comprising: (a) adding the protein to each sample of a set of samples prepared by mixing a first cell culture fluid and a second cell culture fluid at a series of ratios, wherein the first cell culture fluid and the second cell culture fluid are identical, except the first cell culture fluid comprises a cell lysate and the second cell culture fluid is essentially free of the cell lysates; (b) incubating each sample of the set of samples in an air-sealed container (i) having an interior volume of about 1 mL to about 3 mL, and (ii) having less than 100 microliters of headspace; (c) determining the level of protein reduction and/or the redox potential in each sample of the set of samples to determine the reduction susceptibility of the protein based on the level of protein reduction and the redox potential; and (d) generating a standard curve of reduction susceptibility of the protein based on the determined level of redox potential and the determined level of protein reduction for each sample of the set of samples, thereby generating the standard curve of reduction susceptibility of the protein.

In some embodiments, the standard curve generated using the method described herein is used for selecting a protein for further development.

In some embodiments, the method described herein further comprises generating a standard curve of reduction susceptibility of a reference protein. The reference protein can be, for example, a standard protein whose reduction susceptibility has been previous determined to be acceptable for further development.

In some embodiments, the method described herein further comprises comparing the standard curve of reduction susceptibility of the tested protein with that of the reference protein.

In some embodiments, the method described herein further comprises selecting the protein for further development based on the comparison of standard curves of reduction susceptibility of the protein. In some embodiments, the method described herein includes evaluating the developability of a protein based on its reduction susceptibility. In some embodiments, the method described herein includes selecting a protein for further development based on its developability.

### Examples

The invention is further described in the following examples, which do not limit the scope of the invention described in the claims.

### Example 1: Methods of Determining Protein Reduction Susceptibility

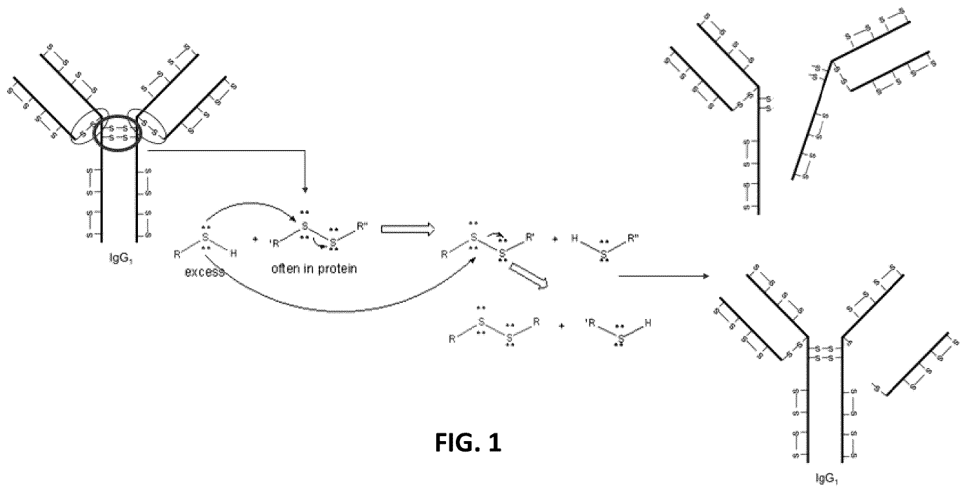

As shown in FIG. 1, reduction of the disulfide bond causes the bond to break, leading to loss of bulk harvest and inability to meet drug substance specifications.

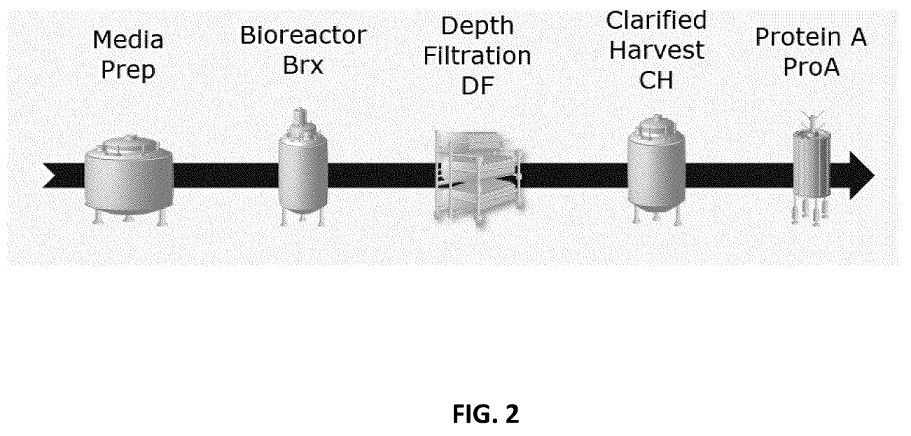

As shown in FIG. 2, reduction occurs mostly in clarified harvest. Lysed cells from apoptosis at the end of culture or caused by shear stress in the harvest release enzymes from the GSH and/or Trx systems leading to a reducing environment in the clarified harvest. Oxygen consumption leads to low DO and a reducing environment in the clarified harvest. Long hold times at elevated temperatures of sealed harvest leads to more GSH/Trx activity, DO consumption and greater risk of Ab reduction.

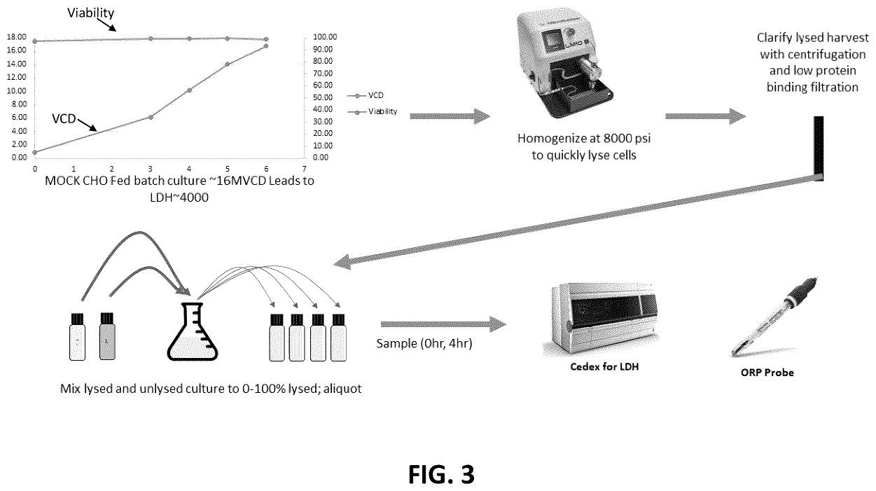

FIG. 3 is a schematic illustration of an experimental outline of an example method for determining the correlation of protein reduction markers, e.g., the level of LDH and ORP. Specifically, cells were cultured to an optimal viable cell density (VCD) while maintaining the viability of the cells. The cells were then harvested and homogenized at 8000 psi to quickly lyse the cells. The homogenized cell lysate was then clarified by centrifugation and passed through a low protein binding filter. The clarified lysed cell culture fluid was then mixed with a culture fluid that is essentially free from cell lysate at different ratios to create a serial dilution.

Specifically, the serial dilution of the cell culture medium was made in 2 mL mini bottles. The lysed cell culture medium was mixed with unlysed culture medium at a series of ratios. The level of LDH was measured via CEDEX (Roche Diagnostics CEDEX BIO LDH, Roche Diagnostics 06343767001). The level of ORP was determined using a laboratory ORP sensor (METTLER TOLEDO).

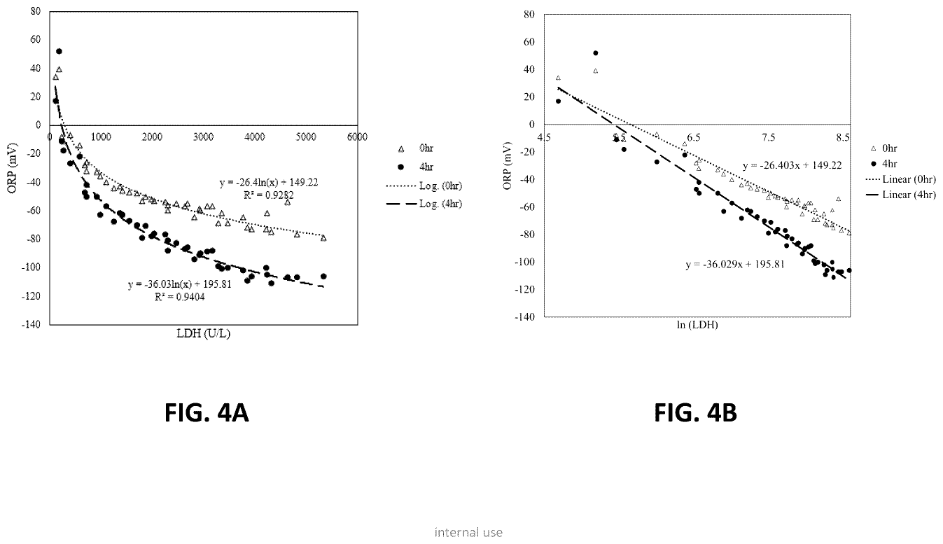

The data in FIGS. 4A-4B establish the correlation between LDH and ORP. The cell culture-based assay allows for the study of the effect on reduction of different process relevant parameters, such as ORP, in culture supernatant. Specifically, ORP was monitored at different LDH values and time points in the cell culture assay. As the amount of lysed material present increases, the more reducing the environment becomes. As shown in FIG. 4B, ORP decreased with increasing LDH and increasing time. This demonstrates that the experimental data are reproducible and consistent, which align with theory underlying the experimental design.

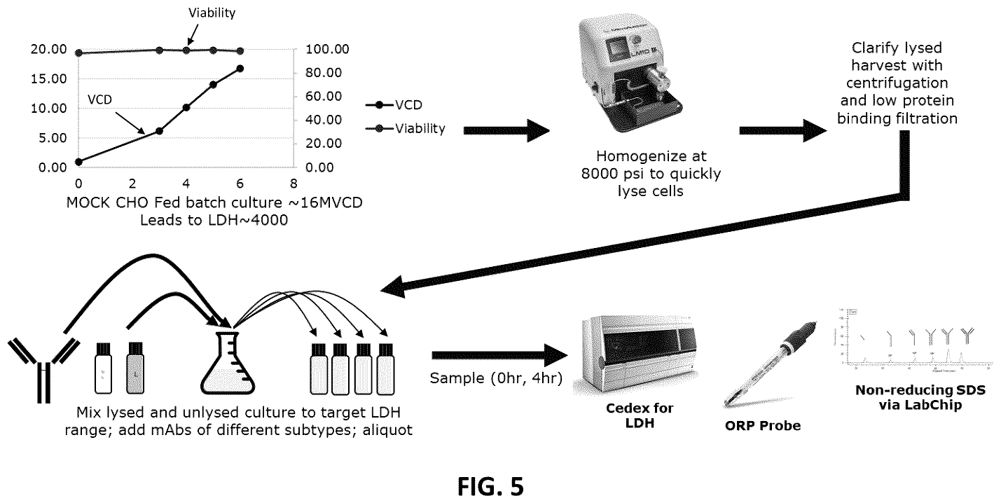

FIG. 5 describes the method used for determining protein reduction susceptibility described herein. Specifically, cells were cultured to an optimal viable cell density (VCD) while maintaining the viability of the cells. The cells were then harvested and homogenized at 8000 psi to quickly lyse the cells. The homogenized cell lysate was then clarified by centrifugation and passed through a low protein binding filter. The clarified lysed cell culture fluid was then mixed with a culture fluid that is essentially free from cell lysate at different ratios to create a serial dilution.

Specifically, the serial dilution of the cell culture medium was made in 2 mL mini bottles. The lysed cell culture medium was mixed with unlysed culture medium at a series of ratios. Each tested protein was added to each mixture of the dilution series and incubated. The liquid was filled to the top of the mini bottles before sealing to create an air-insulated environment. After incubation, the relative levels of intact proteins were measured against the level of LDH in each mini bottle, e.g., at the time point of 4 hours. The level of LDH was measured via CEDEX (Roche Diagnostics CEDEX BIO LDH, Roche Diagnostics 06343767001). The reduction of the proteins (relative levels of intact mAb) was measured via Caliper's LabChip GXII System (Caliper Life Sciences).

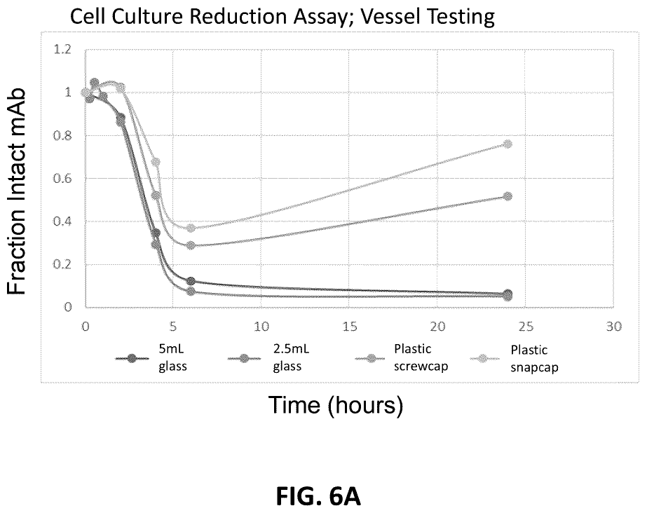

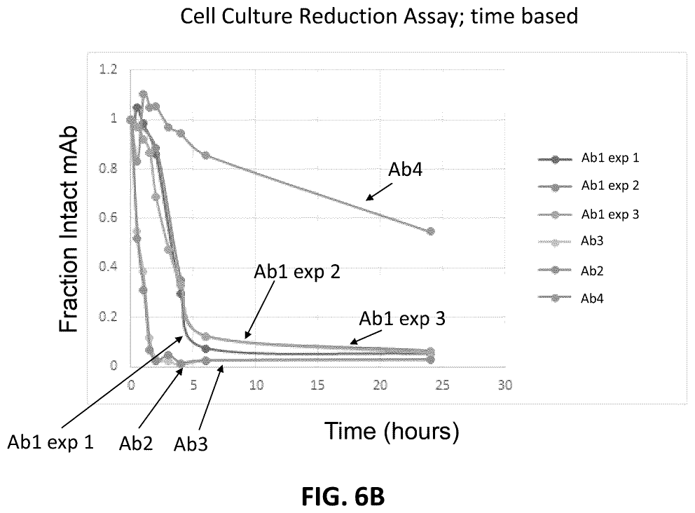

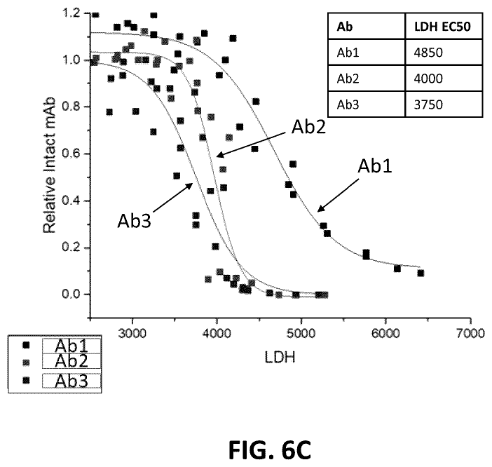

Containers for culture hold are critical. FIG. 6A shows the effect of using different types of containers when performing an exemplary cell culture-based reduction assay. Specifically, different containers were tested in a time-based reduction assay to ensure performance was not impacted with permeability of air over time. As shown in FIG. 6A, glass containers are better than plastic containers for the cell culture-based assay to prevent reoxidation.

Furthermore, a kinetics approach was used to test reduction in lysed culture supernatants (FIG. 6B) with LDH=4000 U/L. Specifically, 7-day CHO culture was harvested and partially lysed by pressure homogenization. Lysed and unlysed culture supernatants were mixed to achieve desired lactate dehydrogenase (LDH) values. Each tested antibody was added and incubated at desired temperature and period of time. Samples were processed for reduction analysis (measured by fraction of intact mAb) by non-reducing SDS gel. Four antibodies (IgG4 (Ab1), IgG1κ (Ab2), IgG1λ, (Ab3), and IgG2 (Ab4)) were tested. These results demonstrate that the assays described herein can provide for an accurate assessment of susceptibility of different antibodies to reduction.

An LDH based culture assay (FIG. 6C) was also performed. As shown in FIG. 6C, as the amount of lysed material present increases, the amount of intact mAb decreases. Specifically, the reduction susceptibility of IgG1λ. (Ab3) is greater than the reduction susceptibility of IgG1κ (Ab2), which is greater than the reduction susceptibility of IgG4 (Ab1). These results demonstrate improved resolution and differentiation of the reduction susceptibility of κ and λ subtypes of immunoglobulins, with a shorter incubation period required (4 h incubation).

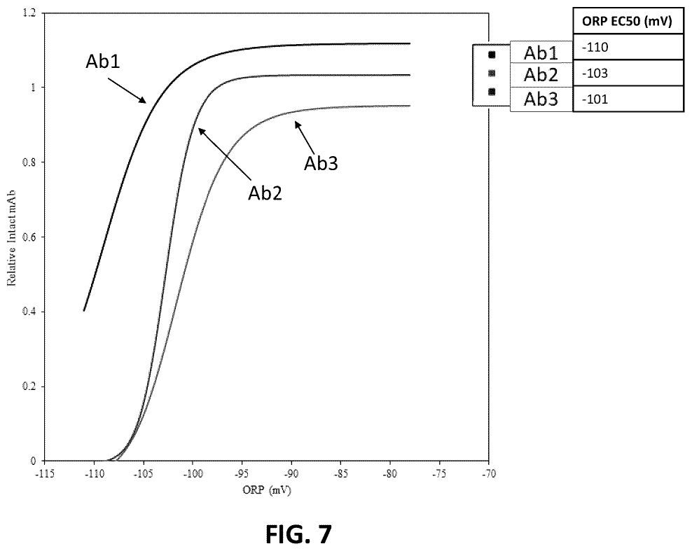

As shown in FIGS. 4A, 4B, and 6C, the correlation between LDH and ORP, and the correlation of LDH and protein reduction were established. Therefore, using LDH as an intermediate, a relationship between ORP and protein reduction was made. As shown in FIG. 7, as the cell culture environment becomes increasingly more reducing (measured by the decrease of the level of ORP), the amount of relative intact mAbs decreases. Specifically, the reduction susceptibility of IgG1λ (Ab3) is greater than the reduction susceptibility of IgG1κ (Ab2), which is greater than the reduction susceptibility of IgG4 (Ab1).

Furthermore, the cell culture-based assay can be used to study the effect of different process parameters, such as dissolved oxygen and temperature shift, on reduction of proteins, which is not possible in chemical assays. The culture-based assay can also be used to study mitigations for reduction such as inhibitors or new process controls, such as controlling ORP above the identified limits for reduction of FIGS. 6A-6C.

The cell culture assay is more process relevant than chemical reduction assays and can be used to study the effects of different culture parameters on reduction, as was done with ORP. Moving forward, these findings can be translated directly to cell culture processes to control reduction; for instance by controlling ORP above the risk limit found for a given antibody in the small scale assay using oxygen addition or chemical additives, or setting LDH-based harvest criteria.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of determining the reduction susceptibility of a protein, comprising:
(a) providing a first cell culture fluid and a second cell culture fluid, wherein the first cell culture fluid and the second cell culture fluid are identical, except the first cell culture fluid comprises a cell lysate and the second cell culture fluid is essentially free of the cell lysate;
(b) mixing the first cell culture fluid and the second cell culture fluid at a series of ratios, thereby preparing a set of samples;
(c) adding the protein to each sample of the set of samples and incubating each sample of the set of samples in an air-sealed container (i) having an interior volume of about 1 mL to about 3 mL, and (ii) having less than 100 microliters of headspace;
(d) determining the level of protein reduction and/or the redox potential in each sample of the set of samples; and
(e) determining the reduction susceptibility of the protein based on the level of protein reduction and/or the redox potential determined for each sample of the set of samples in step (d), thereby determining the reduction susceptibility of the protein.

2. The method of claim 1, wherein the cell lysate was obtained through homogenization of cells.

3. The method of claim 1, wherein the cell lysate was centrifuged to remove cellular debris before step (b).

4. The method of claim 1, wherein the second cell culture fluid is obtained by removing essentially all of the cells from a cell culture medium.

5. The method of claim 1, wherein the protein is a recombinant protein.

6. The method of claim 5, wherein the recombinant protein is an antibody, or antigen-binding fragment thereof.

7. (canceled)

8. The method of claim 1, wherein the level of protein reduction is determined by assessing the oxidation state of the disulfide bonds in the protein.

9. The method of claim 1, wherein the redox potential is determined by measuring one or more of lactate dehydrogenase (LDH), the nicotinamide adenine dinucleotide (NAD+)/reduced NAD+(NADH) redox couple, the NADP+/reduced NADP+(NADPH) redox couple, NADPH, thioredoxin (Trx), and/or glutathione (Grx).

10. The method of claim 1, wherein the redox potential is determined by using a redox potential probe.

11. The method of claim 1, wherein the level of protein reduction is determined by the amount of intact and/or reduced protein after the incubating with each sample of the set of samples.

12. The method of claim 11, wherein the amount of intact and/or reduced protein is determined using a native SDS-PAGE gel or a microfluidic device.

13. (canceled)

14. The method of claim 1, comprising determining the level of protein reduction and the redox potential in each sample of the set of samples and further comprising generating a graph of the protein reduction susceptibility based on the determined redox potential and the determined level of intact or reduced protein for each sample in the set of samples.

15. The method of claim 1, wherein the air-sealed container is an air-sealed glass vial.

16. The method of claim 1, wherein the air-sealed container has an interior volume of about 2 mL to about 2.5 mL.

17. The method of claim 1, wherein the air-sealed container has less than 50 microliters, less than 10 microliters, or 0 microliter of headspace.

18. The method of claim 1, wherein the protein is incubated with each sample of the set of samples for about 0.5 to about 4 hours prior to step (c).

19. The method of claim 1, further comprising comparing the reduction susceptibility of the protein with a reference standard, wherein the reference standard is a standard curve of reduction susceptibility of a reference protein.

20. (canceled)

21. The method of claim 1, further comprising selecting the protein for further development based on the reduction susceptibility of the protein.

22. A method of modifying a culturing condition or cell culture fluid bioprocessing condition of a cell culture medium used for the production of a protein, comprising:
(a) providing a first cell culture fluid and a second cell culture fluid, wherein the first cell culture fluid and the second cell culture fluid are identical, except the first cell culture fluid comprises a cell lysate and the second cell culture fluid is essentially free of the cell lysate;
(b) mixing the first cell culture fluid and the second cell culture fluid at a series of ratios, thereby preparing a set of samples;
(c) adding the protein to each sample of the set of samples and incubating each sample of the set of samples in an air-sealed container (i) having an interior volume of about 1 mL to about 3 mL, and (ii) having less than 100 microliters of headspace;
(d) determining the level of protein reduction and/or the redox potential in each sample of the set of samples;
(e) determining the reduction susceptibility of the protein based on the level of protein reduction and/or the redox potential determined for each sample of the set of samples in step (d); and
(f) modifying one or more culturing conditions or cell culture fluid bioprocessing conditions of the cell culture medium based on the reduction susceptibility of the protein,
thereby modifying the culturing condition or the cell culture fluid bioprocessing condition used for the production of the protein.

23.-56. (canceled)

57. A method of determining the reduction susceptibility of a protein, comprising:
(a) adding the protein to each sample of a set of samples prepared by mixing a first cell culture fluid and a second cell culture fluid at a series of ratios, wherein the first cell culture fluid and the second cell culture fluid are identical, except the first cell culture fluid comprises a cell lysate and the second cell culture fluid is essentially free of the cell lysates;
(b) incubating each sample of the set of samples in an air-sealed container (i) having an interior volume of about 1 mL to about 3 mL, and (ii) having less than 100 microliters of headspace; and
(c) determining the level of protein reduction and/or the redox potential in each sample of the set of samples to determine the reduction susceptibility of the protein based on the level of protein reduction and/or the redox potential,
thereby determining the reduction susceptibility of the protein.

58.-77. (canceled)
